(19) United States
(12) Patent Application Publication
(10) Pub. No.: US 2021/0311056 A1
(43) Pub. Date: Oct. 7, 2021

(54) SELF-ADMINISTERED INFECTION TESTING AND RESULT DETERMINATION

(71) Applicant: Vivera Pharmaceuticals Inc., Newport Beach, CA (US)

(72) Inventors: Paul Edalat, Newport Beach, CA (US); Mehdi Hatamian, Mission Viejo, CA (US)

(73) Assignee: Vivera Pharmaceuticals Inc., Newport Beach, CA (US)

(21) Appl. No.: 17/220,875

(22) Filed: Apr. 1, 2021

Related U.S. Application Data
(60) Provisional application No. 63/018,383, filed on Apr. 30, 2020.
(60) Provisional application No. 63/003,722, filed on Apr. 1, 2020.

(51) Int. Cl. G01N 33/569; A61B 5/15; G01N 21/77

(52) U.S. Cl. CPC G01N 33/56983; G01N 21/77; A61B 5/150786

(57) ABSTRACT

A self-administered tester for infection may include a sample collection element for receiving fluid samples including bodily fluids. The sample collection elements may provide at least a portion of a collected sample to one or more evaluation elements. Each evaluation element may determine whether some evaluation criteria have been satisfied. Result indicators may provide visual indicators showing whether the evaluation criteria were satisfied. The self-administered tester may test for a Severe Acute Respiratory Syndrome Coronavirus-2 (SARS-CoV-2) infection using evaluators associated with the Immunoglobulin G (IgG) antibody and the Immunoglobulin M (IgM) antibody.

### CROSS-REFERENCE TO RELATED APPLICATIONS

This application claims priority to U.S. Provisional Patent Application Ser. No. 63/003,722, filed on Apr. 1, 2020. This application claims priority to U.S. Provisional Patent Application Ser. No. 63/018,383, filed on Apr. 30, 2020.

### BACKGROUND

Since its discovery in December 2019, the Severe Acute Respiratory Syndrome Coronavirus-2 (SARS-CoV-2) virus, the virus that causes Coronavirus Disease 2019 (COVID-19) has become a global pandemic. Cities in almost every state of the United States, as well as other major cities around the world, have ordered citizens to shelter in place or stay at home in a collective effort to contain the spread of the SARS-CoV-2 virus.

SARS-CoV-2 can be spread through airborne transmission of droplets and/or close physical contact. The virus can remain active for up to three days on hard surfaces such as plastic, cardboard, and stainless steel, which makes reducing the incidences of infection difficult.

Laboratory testing for the SARS-CoV-2 virus may take one to five days to achieve results. Further, such testing is typically done at a medical facility and requires involvement of various personnel such as practitioners, lab technicians, etc.

Therefore, there exists a need for a tester that can be used at the home or office and allows users to self-administer a test and receive results without involving other parties.

### BRIEF DESCRIPTION OF THE SEVERAL VIEWS OF THE DRAWING

The novel features of the disclosure are set forth in the appended claims. However, for purpose of explanation, several embodiments are illustrated in the following drawings.

FIG. 1 illustrates an example overview of one or more embodiments described herein, in which a tester provides a result based on a received sample;

FIG. 2 illustrates an example overview of one or more embodiments described herein, in which a tester provides, via a user device, a result based on a received sample;

FIG. 3 illustrates an example overview of one or more embodiments described herein, in which a tester provides, via an analyzer, a result based on a received sample;

FIG. 4 illustrates a schematic block diagram of one or more embodiments of a tester described herein;

FIG. 5 illustrates a front elevation view of a tester of one or more embodiments described herein in an unused state;

FIG. 6 illustrates a front elevation view of a tester of one or more embodiments described herein, indicating a negative test result;

FIG. 7 illustrates a front elevation view of a tester of one or more embodiments described herein, indicating a positive early-stage test result;

FIG. 8 illustrates a front elevation view of a tester of one or more embodiments described herein, indicating a positive late-stage test result;

FIG. 9 illustrates a front elevation view of a tester of one or more embodiments described herein, indicating a positive concluded-stage test result;

FIG. 10 illustrates a front elevation view of a tester of one or more embodiments described herein, indicating an invalid test result;

FIG. 11 illustrates a plot of exemplary response data used to define test thresholds associated with the tester of one or more embodiments described herein;

FIG. 12 illustrates a testing kit of one or more embodiments described herein;

FIG. 13 illustrates a flow chart of an exemplary process that provides a test result using a tester of one or more embodiments described herein;

FIG. 14 illustrates a flow chart of an exemplary process that provides a test result using a tester of one or more embodiments described herein and analyzer of one or more embodiments described herein; and

FIG. 15 illustrates a schematic block diagram of one or more exemplary devices used to implement various embodiments.

### DETAILED DESCRIPTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide a self-administered tester. The self-administered tester may be able to receive a sample or specimen (e.g., blood, saliva, mucous, tears, etc.) associated with a subject or patient. The self-administered tester may be able to test for presence of infections, viruses, diseases, bacteria, antibodies, antigens, and/or other appropriate constituent elements of a sample. The self-administered tester may provide an indication of the test result (e.g., “positive”, “negative”, etc.). The self-administered tester may indicate stage of progression (and/or other appropriate test result information).

In some embodiments, a dedicated analyzer device or system may be able to receive self-administered testers, evaluate tester information to generate a set of results, and provide the results to the subject. In some embodiments, a user device such as a smartphone may execute an application of some embodiments such that the user device and application are able to receive information from the self-administered tester, analyze the information, and provide results to the user.

Throughout this disclosure, although various examples may refer to SARS-CoV-2 and/or other specific examples of diseases, viruses, antigens, etc., one of ordinary skill in the art will recognize that different embodiments may be associated with various different types of diseases, viruses, antigens, etc. Further, although various examples may refer to specific specimen or sample types, such as blood or saliva, one of ordinary skill in the art will recognize that different embodiments may be associated with various different sample types. In addition, although various examples may describe specific elements, such as a specific antibody (and determining whether such antibody is present in a sample), one of ordinary skill in the art will recognize that different embodiments may be associated with various other elements (e.g., other antibodies). Furthermore, although the disclosure may refer to “self-administered” tests, one of ordinary skill in the art will recognize that such that medical practitioners, caregivers, and/or other parties may also administer such tests, as appropriate.

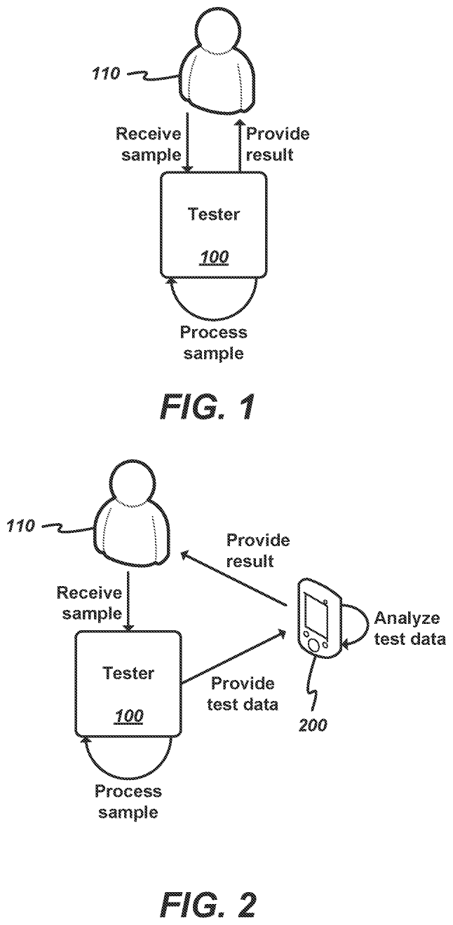

FIG. 1 illustrates an example overview of one or more embodiments described herein, in which a tester or tester device 100 provides a result based on a received sample. As shown, tester 100 may receive a sample or specimen from a user or subject 110.

The sample may be received in various appropriate ways, depending on the sample type, test type, and/or other relevant factors. Samples or specimens may include, for instance, bodily fluids (e.g., blood, saliva, tears, mucous, stool, urine, etc.), tissue or organ samples, etc. For example, some embodiments may collect a blood sample via a collection port of the tester 100. In some embodiments, collected samples may be associated with other data collected via other devices, components, or interfaces (e.g., body temperature of a patient, exhibited symptoms, etc.).

As shown, the tester 100 may process the received sample in various appropriate ways. Processing may include moving portions of a received sample among components of the tester 100, mixing portions of the specimen with various buffers or other agents, exposing portions of the sample to various reactive agents or sensors, waiting designated times for reactions to occur, and/or otherwise manipulating a received sample. For instance, a blood sample may be mixed with a buffer agent.

Processing the sample may include evaluating portions of the sample in various ways. For instance, portions of the sample may be wicked or otherwise transported to one or more evaluation elements associated with the tester 100. As another example, portions of the sample may be exposed to one or more sensors associated with the tester 100.

Each evaluation element may be able to detect one or more antibodies, antigens, and/or other constituent elements of a specimen. In some embodiments, evaluation elements may be able to measure a magnitude or concentration of one or more elements. Such measurement may include determining that an element is present with a specified range of concentrations. In some cases, evaluation elements may be able to produce an output value indicating a concentration or other measure of a constituent element (e.g., a proportional output indicating a concentration percentage of a particular antibody).

As shown, the tester 100 may provide one or more test results to the user 110. Such test results may include visual indicators (and/or other appropriate indicators) that may be easily conveyed to the user 110. For example, some embodiments may provide a set of response areas that each indicate a positive or negative value associated with the test.

For example, a first response area may indicate whether a test result is valid. To be valid, various test criteria or conditions may have to be satisfied. Example of such criteria include sample volume or amount, elapsed processing time, an associated measurement exceeding a minimum or cutoff threshold value, etc. A valid result may be indicated by a bar or line (or other indicator such as a symbol, number, text, graphics, etc.) appearing in the response area. An invalid result may be indicated by a blank or unchanged response area (or other indicator such as a symbol, number, text, graphics, etc.).

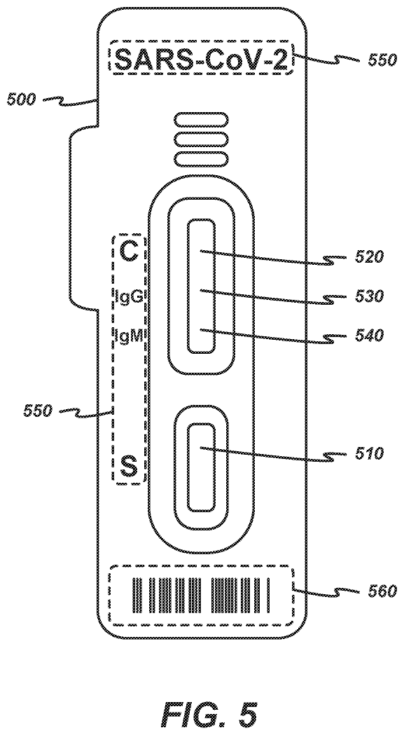

Additional response areas may each indicate whether a particular antibody or antigen is present at a specified concentration. Together, the various response areas may indicate stage of progression and/or other information. Various example responses are described below in reference to FIG. 5-FIG. 10.

FIG. 2 illustrates an example overview of one or more embodiments described herein, in which a tester 110 provides, via a user device 200, a result based on a received sample. The tester 100 may receive and process the sample in a similar manner to that described above.

Tester 100 may provide test data to user device 200. User device 200 may be a device such as a smartphone, tablet, laptop, wearable device, etc. Test data may include data related to the test (e.g., type, output type, criteria, etc.), processing (e.g., applied agent type, if any, elapsed time, etc.), evaluation (e.g., antigen or antibody types), and/or results (e.g., measured values, indications of result ranges, etc.).

Test data may be provided in various appropriate ways across various appropriate communication channels. For instance, test data may be sent across a wireless communication link. As another example, visual or graphical test data may be captured at the user device 200 (e.g., using a camera or other sensor associated with the user device). In some embodiments, user device 200 may collect test data (e.g., subject information, timestamps, tester serial number, test type, etc.). User device 200 may execute an application, access a web page, and/or otherwise provide ways to collect provided test data.

User device 200 may analyze the received data. Such analysis may include, for instance, retrieving information (e.g., from a server or remote storage) based on the test type, tester type, and/or other relevant factors. Data analysis may include comparing received values to any number of threshold values to determine whether a constituent element is within a specified range of values. Data analysis may include mapping visual indicators to a set of possible results (e.g., “negative”, “positive—early stage”, “positive—late stage”, “positive—concluded stage”, etc.) using a resource such as a lookup table and/or sets of reference images. Analysis may include actions such as averaging or other filtering, calculation of aggregate values, comparison to sets of reference data, and/or other types of analysis (e.g., artificial intelligence based on machine learning).

User device 200 may provide the test results (and/or other appropriate information) to the user 110. The result may be provided via the application of some embodiments, a web page, and/or other appropriate resources. The result may be provided using various graphical user interface elements and/or other appropriate elements. For instance, the mapped result (e.g., “positive—early stage”) may be displayed in text. Other relevant information (e.g., measured values, threshold values, confidence in result, etc.) may also be provided, if available. Results may be provided to other resources, such as a server or storage, other user devices, etc. via email, text, push notification, and/or other appropriate ways.

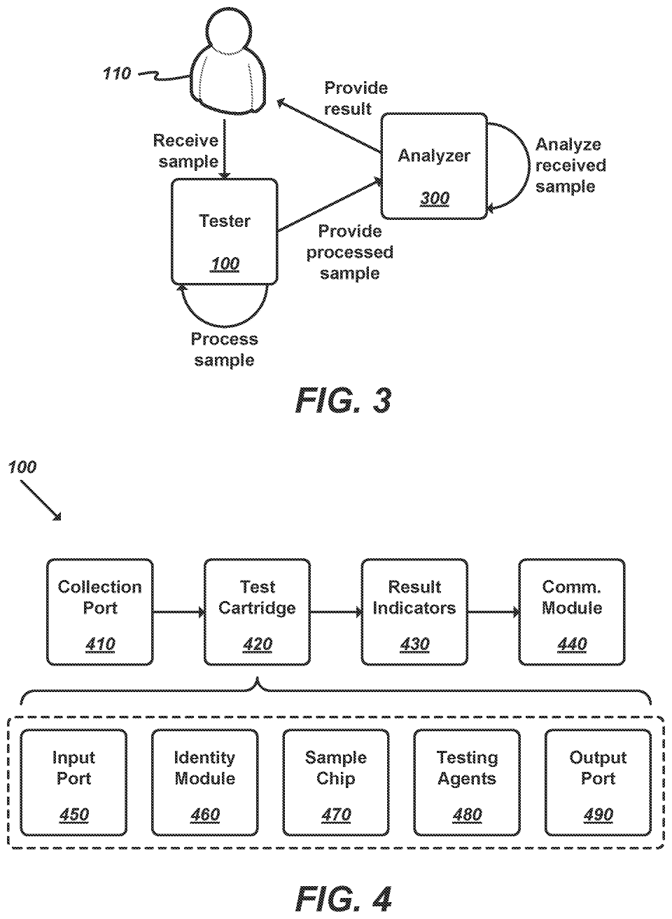

FIG. 3 illustrates an example overview of one or more embodiments described herein, in which a tester 100 provides, via an analyzer, a result based on a received sample. The tester 100 may receive and process the sample in a similar manner to that described above.

In some embodiments, tester 100 may provide the processed sample to analyzer 300. In some embodiments, tester 100 may provide test data to analyzer 300 in a similar manner to that described above in reference to user device 200.

If the processed sample is provided to analyzer 300, the sample may be provided by providing the tester 100, or a portion thereof, to a receptacle of the analyzer 300. In some embodiments, the tester 100 may include a removable cartridge that is able to be received by the analyzer 300. In some embodiments, the tester 100 may include a coupling or connector that is able to transfer sample contents or data to a complementary coupling or connector of the analyzer 300.

Analyzer 300 may be a stand-alone telemedicine device that is able to interact with the tester 100, components thereof, system components such as remote servers or storages, and/or other appropriate elements. In addition, analyzer 300 may include various user interface elements (e.g., a touchscreen, speakers, keypad, cameras, biometric sensors, etc.) that may be able to receive information and/or commands from the user 110 and/or provide information to the user 110. Analyzer 300 may include various network connections and/or communication features that allow for voice and/or video conferencing with one or more medical practitioners or other remote service providers.

Analyzer 300 may analyze the received sample and/or test data. Such analysis may include similar analysis to that described above in reference to user device 200. Analyzer 300 may include various sensors or other evaluation elements that may be able to interact with a received sample, the tester 100, a tester cartridge, and/or other appropriate elements. The tester 100, tester cartridge, and/or other elements may retain collected specimens to be evaluated without contaminating evaluation elements of the analyzer 300. In some cases, and/or under some circumstances, the analyzer 300 may directly contact the received sample, using components that are replaceable, disposable, and/or that are able to be sterilized or otherwise appropriately prepared for use on another sample.

Analyzer 300 may provide the test results to the subject 110. Such results may be provided in various appropriate ways, including graphical indicators, audio indications, etc. The results may be communicated to a user device 200 or other resource via email, text message, push notification, and/or other appropriate ways. Results may be provided to resources such as an external server or storage associated with a medical facility, work site, etc.

FIG. 4 illustrates a schematic block diagram of one or more embodiments of a tester 100. As shown, tester 100 may include a collection port 410, test cartridge 420, result indicators 430, and communication module 440. The tester 100 may typically include a rigid or semi-rigid body or housing (e.g., a plastic housing). The tester 100 may include various removable covers, stickers, seals, or similar features that may provide access to the various components of tester 100.

Collection port 410 may include an opening, cavity, receptacle, or other such feature that may receive a specimen or sample (e.g., blood, mucous, saliva, sputum, tears, etc.). In some embodiments, the collection port 410 may include automatic collection elements such as a lancet, pump, etc. that may be used to collect a sample.

Test cartridge 420 may include elements such as an input port 450, identity module 460, sample chip 470, testing agents 480, and output port 490. Test cartridge 420 may include a housing that is able to contain fluids or other samples. For instance, the test cartridge 420 may include a self-sealing silicone housing. In this way, the test cartridge 420 may be able to be removed and/or provided to a component such as analyzer 300 without contaminating the sample (or contaminating evaluation elements intended to be used across multiple samples).

Input port 450 may include various inlets, openings, conduits, couplings, etc. that may allow a sample to be received from the collection port 410. Input port 450 may self-sealing and/or include a valve or similar element such that a collected sample is not able to exit the input port 450.

Identity module 460 may provide information related to the tester 100 and/or other test data. Such information may be provided via elements such as graphic codes, NFC tags, etc. Information provided by the identity module 460 may include, for instance, test type, tester serial number or other ID, test parameters, instructions, etc. In some embodiments, identity module 460 may include, or have access to, a memory that may allow for updating stored information such as cartridge status (e.g., “unused”, “sample collected”, “results provided”, etc.), subject information (e.g., name, patient ID, anonymized ID, etc.), timestamps, and/or other appropriate information.

Sample chip 470 may include various evaluation elements that may be used to analyze a collected sample. Such evaluation elements may include, for instance, paper or fabric treated with and/or otherwise exposed to various chemical solutions that react when contacted by a sample that meets some evaluation criteria. In some embodiments, evaluation elements may include chambers or reservoirs that are at least partially filled with such chemical solutions that react when contacted by a sample that meets some evaluation criteria. In some embodiments, such evaluation elements may generate visual indicators that may serve as result indicators 430. Sample chip 470 may include various cavities, conduits, valves, etc. that may be used to store or transport various testing agents 480, collected samples (or portions thereof), and/or mixtures thereof.

Testing agents 480 may include, for instance, buffer solutions, thickening agents, thinning agents, attraction potential for hydrogen atoms (pH) balancing agents, etc. Testing agents 480 may include evaluation solutions that may react to various constituent elements of interest, such as antibodies and/or antigens.

Output port 490 may include various outlets, openings, conduits, couplings, etc. that may allow a sample (or processed sample) to be passed to the result indicators 430 and/or other elements of tester 100. Output port 490 may self-sealing and/or include a valve or similar element such that a collected sample is not able to unintendedly exit the output port 490.

Result indicators 430 may include various indicator elements, such as paper or fabric treated with and/or otherwise exposed to various chemical solutions that may generate a visual indicator when contacted by a sample that meets some evaluation criteria. In some embodiments, result indicators 430 may include recesses, chambers, and/or reservoirs that are at least partially filled with such chemical solutions that generate a visual indicator contacted by a sample that meets some evaluation criteria. Such recesses, chamber, and/or reservoirs may be at least partly visible via clear “windows” or other viewing features associated with the tester 400. For instance, a positive result may cause a bar indicator or other symbol associated with a result indicator 430 to become visible. Result indicators 430 may be associated with various evaluation profiles. Such evaluation profiles may include, for instance, reference images of various results that may be compared to the reference indicators 430. Result indicators 430 may include elements such as graphic codes, bar codes, and/or other digital codes that may be analyzed by an analyzer 300, user device 200, and/or other appropriate resource to match the codes to various results (e.g., using a lookup table). Result indicators 430 may be able to indicate among discrete ranges (e.g., by including one to five or one to ten bars to indicate concentration percentage along a specified range of values) or provide granular changes in output (e.g., by providing a darker symbol or more highly saturated color output to indicate a measured value or range.

Communication module 440 may include elements such an NFC tag or Bluetooth transceiver that are able to communicate with various external devices, components, or systems. Communication module 440 may be able to send test information, tester information, and/or result information to external devices such as analyzer 300, user device 200, etc. The communication module 440 may include various connectors, contacts, couplings, etc. that may allow the communication module 440 to be communicatively coupled to external components such as analyzer 300, user device 200, etc.

Tester 100 may include various pumps, valves, mixers, and/or similar fluid manipulation elements that may interact with the exterior of test cartridge 420 (or sample chip 470) and be reusable across multiple cartridges 420.

Although various specific components are described in reference to tester 100, test cartridge 420, etc., one of ordinary skill in the art will recognize that such components may be arranged in various different ways, as appropriate. For instance, some embodiments may include multiple test cartridges 420 with the listed components distributed among the cartridges. As another example, components included in cartridge 420 in this example may be located externally to the cartridge 420 in some embodiments.

FIG. 5 illustrates a front elevation view of a tester 500 in an unused state. Tester 500 is a specific example of tester 100, and is associated with a SARS-CoV-2 test in this example. As shown, tester 500 may include specimen area 510, response areas 520, 530, and 540, labeling 550, and/or other informational or communicative elements 560. Tester 500 may include various chemicals and/or substances that interact with collected sample materials (e.g., human blood, saliva, mucous, etc.) to determine the presence of various antibodies that indicate whether a test subject has been exposed to an antigen.

Specimen area 510 may be used to collect a specimen to be evaluated. Specimen area 510 may include an opening or other receptacle that may be able to receive a sample or specimen.

Response area 520 may be a “control” response area that is used to indicate whether a sufficient amount of sample material was collected via specimen area 510, whether the proper type of specimen was deposited within specimen area 510, and/or other relevant attributes of the specimen are acceptable. If a proper specimen and proper amount of specimen were deposited within specimen area 510, and proper testing instructions were followed, an indicator line, mark, or other indicia may appear in response area 520 to indicate that the testing procedure was performed properly and/or any required conditions were satisfied. If the testing procedure was performed improperly and/or there were other possible errors in the operation of the test, an “improper” indicator (or lack of proper indicator) may appear in response area 520. For example, a bar or line indicator may be used to indicate a valid test and lack of such an indicator may be used to indicate an invalid test. As another example, in some embodiments an “X” symbol may indicate an improper or invalid test result, while an “O” symbol may indicate that any required test conditions have been satisfied. Such required test conditions may include specified test criteria such as minimum amount of collected sample, testing procedure (e.g., by evaluating a sample to determine whether a testing agent was mixed with the sample), minimum wait time, environmental attributes (e.g., temperature, humidity, etc.), and/or other appropriate test validity criteria or test conditions.

Response area 530 may indicate the presence of an antibody in the specimen received via the specimen area 510. In this example, response area 530 indicates the presence of the Immunoglobulin G (IgG) antibody. IgG is the main antibody in the human body that helps to battle bacterial and viral infections.

Response area 540 may indicate the presence of another antibody in the specimen received via the specimen area 510. In this example, response area 540 indicates the presence of the Immunoglobulin M (IgM) antibody. IgM is the first antibody to appear in the response to initial exposure to an antigen.

Labeling 550 may include information related to the test type, antibody or antigen of interest, etc. Labeling 550 may further include various indicators, instructions, etc. that may provide usage directions, result keys, etc.

Informational or communicative elements 560 may include graphic codes, such as bar codes or quick-response (QR) codes. In some embodiments, communicative elements 560 may include features such as near field communication (NFC) tags and/or other wireless tags (e.g., Bluetooth) that may be used to provide information related to tester 500. Such information may include, for instance, test type, serial number or other identifying information, test parameters or conditions, and/or other relevant information. In some embodiments, informational or communicative elements 560 may include links to other resources, such as web sites or other online resources associated with the tester 500.

During use, a sample or specimen may be collected in various appropriate ways. For instance, a lancet may be used to prick a finger of a test subject or patient to obtain a small amount of blood. The small amount of blood, e.g., one or two drops of blood, may be collected via specimen area 510. A buffer solution may be applied to (and/or included at) specimen area 510 to sufficiently wet the specimen area 510, and may assist in wicking of the specimen (e.g., blood) to response areas 520, 530, and/or 540. Response areas 520-540 may each include various chemicals that respond to one or more different constituents in the specimen placed in the specimen area 510. As the specimen from specimen area 510 is wicked and/or otherwise drawn into response areas 520-540, indications of the presence or absence of various constituents of the specimen may be indicated.

After a specified amount of time (e.g., twenty minutes, thirty minutes, etc.), tester 500 may have sufficient time to respond to the constituents in the specimen received via specimen area 510. Because tester 500 can be easily used and read by a patient himself, the associated test may be performed at home rather than exposing a potentially contagious patient to others in a medical office, urgent care facility, drive-up testing site, etc., which may reduce the spread of a particular contagious virus and/or bacteria. Tester 500 may also reduce costs associated with contagious disease testing, as well as providing results in near-real-time.

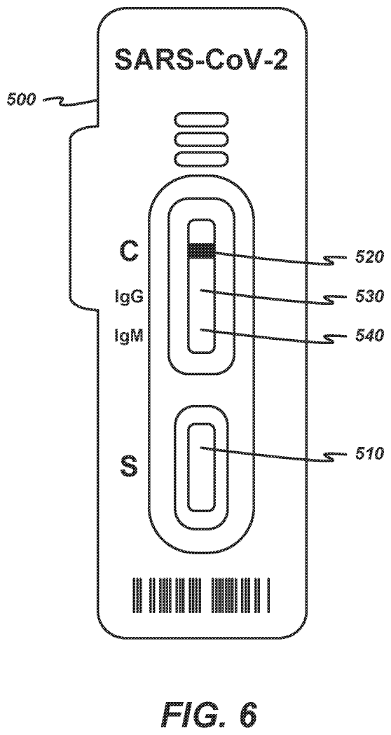

FIG. 6 illustrates a front elevation view of a tester 500 of one or more embodiments described herein, indicating a negative test result. When a sufficient amount of a specimen is received via specimen area 510, and sufficient time has elapsed, the specimen will have wicked or otherwise been drawn toward response areas 520-540. As shown in this example, response area 520 includes a bar indicator that indicates any required testing conditions have been satisfied. Various other indicators, such as symbols, changes in color, etc. may be used to indicate a valid or invalid test result. In this example, the bar indicator at response area 520 indicates that the test conditions for this particular specimen were satisfied. The lack of indicators in response areas 530-540 indicates the lack of IgM or IgG antibodies in the specimen, and thus the response to this particular specimen indicates that the tested-for antibodies (IgM and IgG) are not present in the sample, or do not exceed a minimum detection threshold value.

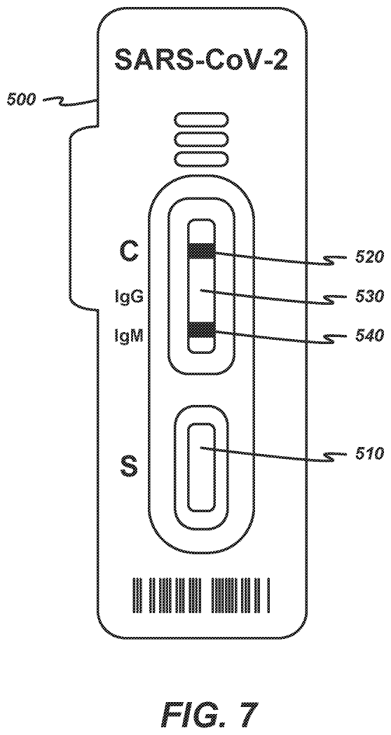

FIG. 7 illustrates a front elevation view of a tester 500 of one or more embodiments described herein, indicating a positive early-stage test result. As shown in this example, response area 520 includes a bar indicator that indicates any required testing conditions have been satisfied. Further in this example, the bar indicator at response area 540, and lack of such a bar indicator at response area 530 indicates that the sample received via specimen area 510 includes IgM antibodies, but does not include IgG antibodies (or the measured IgG antibodies do not exceed a minimum detection threshold value). Such a result indicates an “early-stage” response to the tested-for virus, which in this example may indicate that the test subject was exposed to the tested-for virus approximately four to ten days prior to the test being performed.

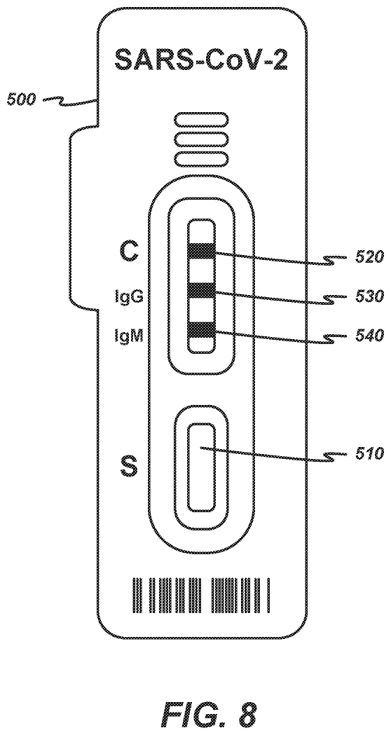

FIG. 8 illustrates a front elevation view of a tester 500 of one or more embodiments described herein, indicating a positive late-stage test result. As shown in this example, response area 520 includes a bar indicator that indicates any required testing conditions have been satisfied. Further in this example, the bar indicator in both response areas 530 and 540 indicates that the sample received via specimen area 510 includes IgM antibodies and IgG antibodies. Such a result indicates a “late-stage” response to the tested-for antigen or virus, which in this example may indicate that the test subject was exposed to the tested-for virus approximately eleven to twenty-four days prior to the test being performed.

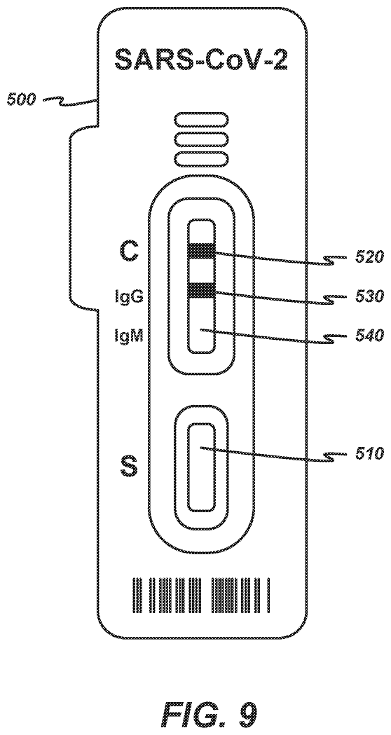

FIG. 9 illustrates a front elevation view of a tester 500 of one or more embodiments described herein, indicating a positive concluded-stage test result. As shown in this example, response area 520 includes a bar indicator that indicates any required testing conditions have been satisfied. Further in this example, the bar indicator at response area 530 and lack of such a bar indicator at response area 540 indicates that the sample received via specimen area 510 includes IgG antibodies, but does not include IgM antibodies (or the measured IgM antibodies do not exceed a minimum detection threshold value). Such a result indicates a “concluded-stage” response to the tested-for antigen or virus, which in this example may indicate that the test subject was exposed to the tested-for antigen or virus longer than twenty-four days prior to the test being performed. In some cases, such a result may indicate that the test subject has developed an immunity to the tested-for virus or antigen.

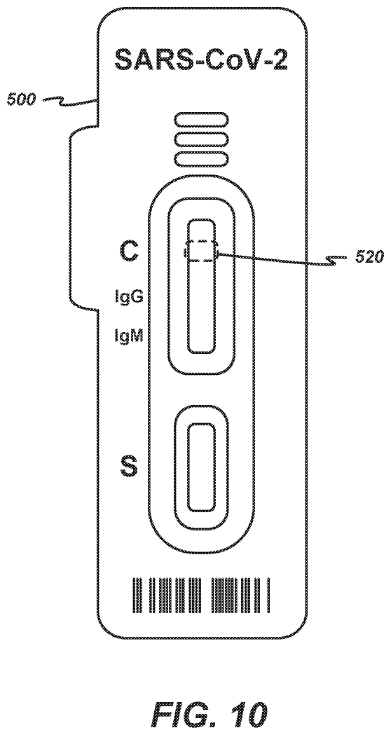

FIG. 10 illustrates a front elevation view of a tester 500 of one or more embodiments described herein, indicating an invalid test result. As shown in this example, response area 520 does not include an indicator, thus indicating that there is something improper or invalid about the result shown by tester 500 (if any). Such an invalid or improper result indication may indicate that some required test condition (e.g., amount of collected sample, testing procedure, wait time, etc.) has not been satisfied. In some embodiments, response area 520 (and/or other similar features) may provide an indication of which test conditions have been satisfied or not. For example, an “X” symbol may indicate that the amount of collected sample was not sufficient, an “O” symbol may indicate that testing procedures were not followed (e.g., if a sample is not mixed properly with a buffer solution), “blank” response area 520 may indicate any or all test conditions were not satisfied, and the bar symbol may indicate that all test conditions have been satisfied.

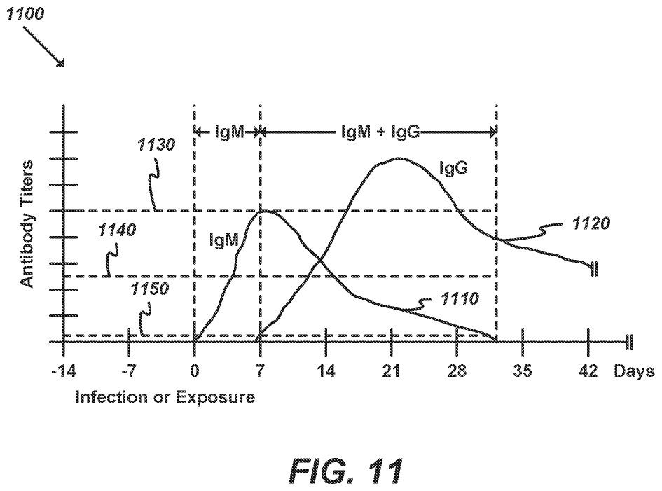

FIG. 11 illustrates a plot 1100 of exemplary immunoglobulin response to infection data used to define test thresholds associated with the tester 500. Plot 1100 shows the build-up of antibodies in humans on the y-axis versus time of infection or exposure on the x-axis. IgM antibodies are represented by series 1110 and IgG antibodies are represented by series 1120. As shown, the IgM antibodies build up and peak at approximately seven days after infection. The IgG antibodies begin to build up approximately seven days after infection and peak at approximately twenty-one days after infection. Tester 500, depending on internal chemistry (and/or other relevant attributes), can test for a desired amount of IgG and IgM antibodies, and report the desired amount (i.e., a concentration over a certain amount) in reporting areas 530 and 540. By selecting the percentage of presence of IgG and IgM antibodies, tester 500 can determine when, if ever, a particular patient was infected with the tested-for virus/bacteria. The IgM and IgG assays (i.e., reporting areas 530 and 540) are thus able to determine infection time (how long the patient has been sick) from four to thirty-two days at a sensitivity of seventy to one hundred percent depending on infection length. Other types of tests, such as Polymerase Chain Reaction (PCR) testing, do not indicate patient infection stage and may not be specific to SARS-CoV-2. Additionally, recovered patients may have false positive results with PCR tests, adding confusion and unnecessarily expending limited health care resources.

The percentage ranges related to each infection state which depend on the details of the particular chemistry used in the tester 500 are as follows in this example infection profile:

Stage I (early infection, ˜day 4-10): 70%<IgM<90% and IgG<70%;

Stage II (late infection, ˜day 11-16): 70%<IgG<90% and IgM>70%;

Stage III (late infection, ˜day 17-28): 90%<IgG and IgM>cutoff threshold (e.g., 50%);

Stage IV (late infection, ˜day 29-32): 70%<IgG<90% and IgM>cutoff threshold; and

Stage V (infection concluded, ˜day 32 onward): IgG>70% and IgM<cutoff threshold.

The various percentages used in this example are indicated by line 1130 (90%), line 1140 (70%), and line 1150 (cutoff or minimum detectable threshold).

Different infection profiles may include various different stage definitions, numbers of stages, etc., as appropriate. Such infection profiles may be generated based on data collected across multiple subjects, default values, experimental results, and/or other appropriate data. Infection profiles may be at least partly based on measurement and/or indication capabilities of various evaluation elements.

Different numbers of result indicators 430 may allow for profiles including various numbers of outputs or output combinations. Using the example tester 500 and result areas 530-540, result indicator 530 may be associated with an IgG concentration above 70% and result indicator 540 may be associated with an IgM concentration above 70%. Following that example, the above stage I early infection would be indicated by a bar appearing at result area 540 and no indicator at result area 530. Continuing the example, stage II late infection would be indicated by a bar appearing at result area 530 and result area 540. Further continuing the example, stage III late infection would be indicated by a bar appearing at result area 530 and no indicator appearing at result area 540. Further continuing the example, stage IV late infection and stage V infection concluded would also be indicated by a bar appearing at result area 530 and no bar appearing at result area 540. To allow for differentiation between stages III-V in this example, additional result areas may be included (e.g., a result area associated with a concentration of IgM above the cutoff threshold and a result area associated with a concentration of IgG above 90%).

While PCR tests may be able to determine pre-symptomatic infections, a majority of the immediate crisis is due to symptomatic patients and the possibilities of community infection.

Point of care tests, such as tester 100 and/or tester 500, are advantageous for triage environments and mass testing which are both vital methodologies for controlling widespread outbreaks of infection. Because a tester 100 such as tester 500 does not require specialized equipment or training, tester 500 is easier to implement in wide-spread applications such as pandemic outbreaks. Further, because of the short delay in obtaining results, it may be easier for the medical teams to rapidly determine proper treatment plans for infected and non-infected patients.

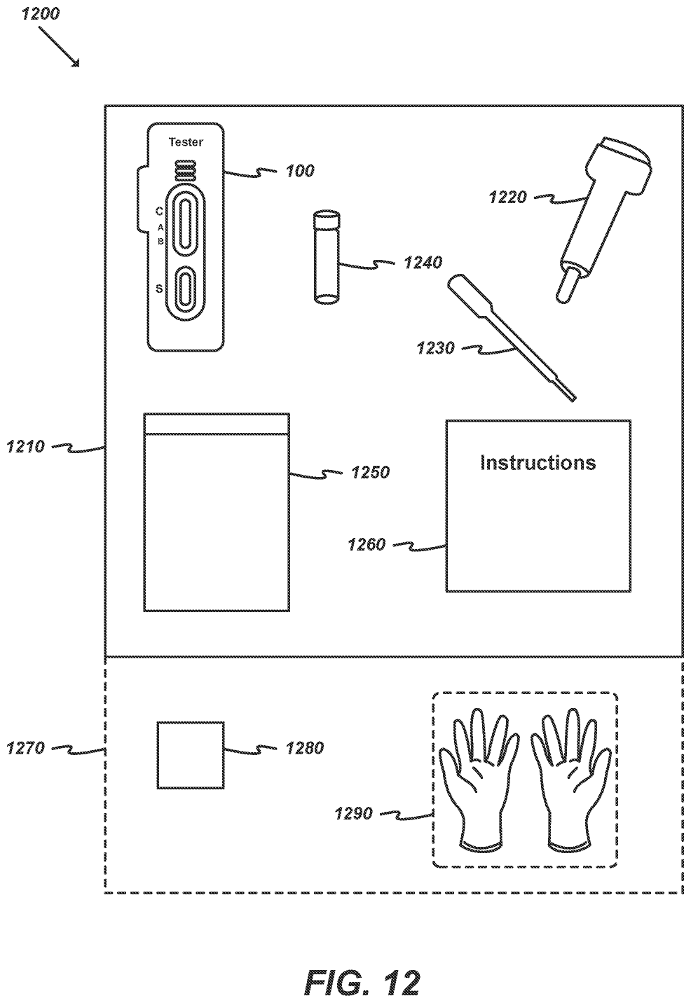

FIG. 12 illustrates a testing kit 1200 of one or more embodiments described herein. As shown, testing kit 1200 may include group 1210, which may include one or more testers 100, lancet 1220, pipette 1230, buffer solution 1240, bag 1250, and instructions 1260. Testing kit 1200 may include at-home group 1270, which may include additional components such as an alcohol pad 1280, gloves 1290, etc.

Testing kit 1200 may be Food and Drug Administration (FDA) approved. Testing kit 1200 may be in compliance with the European Conformity (CE) standard. The CE certification mark indicates conformity with health, safety, and environmental protection standards for products sold within the European Economic Area (EEA), although the CE mark may also be found on products sold outside the EEA, e.g., in the United States, etc., that have been manufactured to EEA standards. An at-home testing kit 1200, which may include at-home group 1270, may also meet FDA and/or CE certifications.

Tester 100 may be able to receive samples or specimens, such as bodily fluids, from a subject and may be able to determine the presence and/or absence of certain constituents in the sample received at tester 100. Although examples described herein may discuss identifying the presence of IgG and IgM antibodies in human blood, tester 100 may detect other antibodies, white blood cell counts, and/or other relevant constituents and/or may be able to receive other types of samples, such as saliva, urine, sputum, tears, mucous, etc., as desired. Any number of testers 100 (e.g., two, four, ten, etc.) may be included in testing kit 1200, as appropriate for phased testing over a period of time.

Lancet 1220 may be optionally included in testing kit 1200 when tester 100 is adapted to use blood as the specimen to be tested. Lancet 1220 may be a simple needle or sharp object used to pierce skin or otherwise allow access to blood from a subject, or may be a spring-loaded device that punctures the skin in a rapid manner to avoid apprehension and pain associated with piercing the skin. Lancet 1220 may be of any type or configuration that is able to access a blood sample for use with testing kit 1200.

Pipette 1230 may be optionally included in testing kit 1200 to transfer buffer solution 1240 from a vial or other container to tester 100. Pipette 1230 may not be necessary in testing kit 1200 if buffer solution 1240 can be effectively transferred to tester 100 in such a manner that a proper test of the collected specimen can be performed by tester 100. For example, and not by way of limitation, if buffer solution 1240 is contained in a “tear-away” packet such that a stream of buffer solution 1240 can be adequately directed toward a desired portion of tester 100, pipette 1230 may not be necessary for inclusion in testing kit 1200. However, FDA/CE certifications may require that a pipette and/or other transfer device for buffer solution 1240 be included in testing kit 1200 and/or testing kit 1270.

Buffer solution 1240 may be an aqueous solution comprising a mixture of a weak acid and a weak conjugate base. The pH may change very little when a small amount of strong acid or base is added to buffer solution 1240. As such, buffer solution 1240 may keep the pH of a bodily fluid sample at a nearly constant value across a wide range of possible fluid additions. Further, buffer solution 1240 may wick the bodily fluid and/or saturates the absorbent portion of tester 100 with enough of the bodily fluid to be tested to bring the bodily fluid to the chemically reactive portions of tester 100 in a controlled manner. Depending on the bodily fluid to be tested by tester 100, buffer solution 1240 may include different acids and/or bases to coordinate with the tester 100 design and the specimen type being examined.

Bag 1250 may be optionally included in testing kit 1200 for disposal of biologically hazardous materials in testing kit 1200 and/or at-home kit 1270. Bag 1250 may have markings indicating that the contents of bag 1250 are biohazardous waste, and may also have instructions indicating how to properly dispose of bag 1250 and any contents. Bag 1250 may include a seal (e.g., a zipper lock seal or an adhesive seal) such that biologically hazardous materials and/or other refuse are securely retained by bag 1250.

Instructions 1260 may be optionally included in group 1210 and/or at-home group 1270, and may indicate how to use, testing methodology, and order of performance of steps for the testing kit 1200. In some embodiments, instructions 1260 may be printed on the packaging of testing kit 1200, rather than being a separate sheet as shown. Further, in some embodiments, instructions 1260 may include different content depending on whether the kit 1200 is intended for medical personnel use or personal use, whether the kit 1200 is intended to achieve FDA and/or CE certified, or other reasons.

At-home group 1270 may include additional components to allow the at-home testing kit 1200 to be a “stand-alone” or “all-in-one” collection of components. In such embodiments, all of the components used in a single administration of tester 100 may be included in at-home kit 1200. In other embodiments, at-home testing kit 1200 may not include all of the components used in a single administration of tester 100; some components that are not included may be listed on the packaging for at-home kit 1200 such that a consumer can determine whether or not they already have such components available.

For example, and not by way of limitation, at-home kit 1200 may include tester 100 and buffer solution 1240. Such an at-home kit 1200 may be purchased as a “first time” kit, or as an “extra” kit because consumers either already have the remaining items needed to properly perform the testing and/or because consumers previously purchased a testing kit 1200 that included the remaining components used in properly performing a test. Other combinations of components of a testing kit 1200 are possible without departing from the scope of the present disclosure.

In an aspect of the present disclosure, there may be a correlation between the tester 100 and the buffer solution 1240 for a given bodily fluid. As such, a testing kit 1200 may include both the tester 100 and the corresponding buffer solution 1240. Some testers 100 may not require a buffer solution 1240 and/or may be operational with any number of buffer solutions 1240 or without a buffer solution 1240 altogether. The contents of the testing kit or at-home testing kit 1200 may include any number of items depending on what a particular consumer may already have, the anticipated end use of the testing kit 1200, and/or other factors.

In an “all-in-one” at-home kit 1200 including at-home group 1270, an alcohol pad 1280 and/or gloves 1290 (e.g., medical disposable gloves such as latex or rubber gloves) may be included to provide a subject with all of the implements needed to properly perform a test using tester 100. For the most accurate results, tester 100 may require that the site where blood is to be drawn to be sterile. As such, an alcohol pad 1280 can be used to wipe the site prior to the blood draw. Contamination from skin oils or other contaminants may also affect the test and/or accuracy; as such, gloves 1290 may be worn during the testing procedure that uses tester 100 and/or other portions of testing kit 1200.

In some cases, testing kit 1200 may be available by prescription only. For example, and not by way of limitation, testing kit 1200 may be prescribed by a medical practitioner to a patient that exhibits certain symptoms. The patient may then visit a pharmacy to acquire testing kit 1200 or the patient may be administered a test using testing kit 1200 at a facility such as a medical practitioner's office.

If the test associated with the testing kit 1200 is administered at the medical practitioner's office, the medical practitioner may administer the test and use the various components of the testing kit 1200, and discuss the results with the patient face-to-face. However, if the patient visits the pharmacy for a prescription-ordered kit to self-perform the testing using at-home testing kit 1200, a tele-health session (e.g., telephone call, video call, video conference, instant messaging, etc.) may be held between the medical practitioner and the patient.

In an aspect of the present disclosure, the tele-health session may be held to assist the patient in performing the test using testing kit 1200. In such cases, the medical practitioner may ensure that the patient is properly performing the test. In some embodiments, the tele-health session may only include the reading and/or interpretation of the results (e.g., via photo or video, by having the patient describe the results to the medical practitioner using voice and/or text, etc.) by the medical practitioner, along with any medical advice that the medical practitioner may have with respect to the interpreted results of the test.

In some embodiments, testing kit 1200 may be available without a prescription from a medical practitioner as an “over-the-counter” device. A consumer may purchase testing kit 1200 at a retailer, in person or via online shopping, and perform the test themselves with or without medical practitioner assistance and/or interpretation. In such cases, instructions 1260 may also include test result explanations to aid the person in interpreting the results given by tester 100. Instructions 1260 may also include additional warnings, etc. to the person who self-administers the test that they should contact their medical practitioner, emergency services such as 911, and/or other persons to contact should there be questions and/or concerns about the testing and/or results delivered.

Testing kit 1200 may or may not have achieved FDA and/or CE certification and/or may only be approved for emergency use only by the FDA and/or CE. Further, testing kit 1200 may not require FDA and/or CE certification depending on the type of test being performed. In such cases, testing kit 1200 may be purchased and self-administered by a consumer, and the results may be optionally shared with one or more medical practitioners as desired.

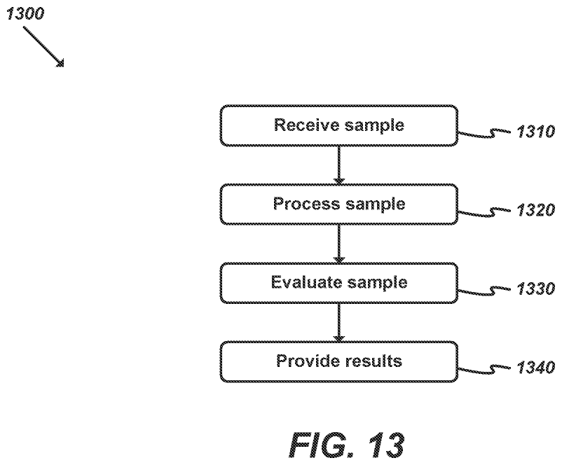

FIG. 13 illustrates a flow chart of an exemplary process 1300 that provides a test result using tester 100. The process may be performed when a possible exposure occurs, at regular intervals, and/or other appropriate circumstances. In some embodiments, process 1300 may be performed by, or using, tester 100. A patient or subject may obtain a tester 100 or testing kit 1200 via prescription or over the counter, as appropriate.

As shown, process 1300 may include receiving (at 1310) a sample or specimen. The sample may be received in various appropriate ways, depending on the type of sample (e.g., blood, saliva, mucous, tears, etc.). For example, a blood sample may be received via specimen area 510.

Process 1300 may include processing (at 1320) the received sample. Such processing may include mixing various substances or solutions with the received sample, such as adding a buffer solution to a blood sample.

The process may include evaluating (at 1330) the received sample. Evaluation may include wicking, drawing, or otherwise providing at least a portion of the sample (or processed sample, if any processing was performed) to one or more evaluation elements, such as response areas 520-540. In some embodiments, various evaluation elements may be moved or manipulated such that the evaluation elements are able to interact with the received sample at the collection point (e.g., specimen area 510). Each evaluation element may include various reactive chemical substances, sensors, and/or other features that may be able to determine various properties associated with the received sample.

Different embodiments may include various different types of evaluation elements with various measurement capabilities. For instance, some embodiments may include binary evaluation elements able to detect whether a constituent element of a received sample exceeds a specified concentration. As another example, some embodiments may include discrete evaluation elements able to detect whether a constituent element is within a particular range from a set of ranges associated with the evaluation element (e.g., an evaluation element may indicate a concentration as an integer value from one to five). Some embodiments may include evaluation elements that are able to detect or measure a concentration of a constituent element across a range of values (e.g., an evaluation element may indicate a concentration as a percentage value).

As shown, process 1300 may include providing (at 1340) test results. Results may be provided in various appropriate ways, such as via a set of visual indicators. Different types of tests, and associated evaluation elements, may be associated with various different types of outputs (e.g., visual indicators, digital or analog signals, etc.) that may indicate the results of each evaluation. For example, some embodiments may present binary results using a bar indicator (e.g., a bar or line may appear to indicate a positive result and no line or bar may indicate a negative result). As another example, if an evaluation element is able to measure across a range of values, a magnitude of the measured value may be indicated using a set of lines (e.g., a set of up to five lines, with one line indicating a minimum value and five lines indicating a maximum value).

Depending on the results of the test, and/or patient circumstances or attributes (e.g., elderly, underlying health conditions, known exposure to the tested-for virus/bacteria, etc.), the patient may self-quarantine between the administration of a first test and the administration of a second test, even if the results of the first test are negative. Depending on the results, or the nature of the test (e.g., if the test was prescribed by a medical practitioner), the patient may consult with a medical practitioner for additional information, advice, etc.

Because each testing kit 1200 may include at least two testers 100, medical providers and other health officials may be able to better understand the rate of spread of a particular virus/bacteria/disease, and to better allocate resources based on the reporting of multiple testing phases in various patients. Further, other relevant information such as rates of recovery may be determined through multiple phases of testing of individual patients.

For example, when a patient receives the test kit 1200, two testers 100 may be included. The patient may administer a first test on the day the test kit 1200 is received using a first tester 100. Depending on the results displayed by the first tester 100, the patient may administer a second test using a second tester 100 at some later time, such as between four and ten days after the first test was administered. This phased testing protocol may illustrate the progression of the infection for a patient which may indicate various different directives available to the medical provider (not infected, quarantine, partial immunity, etc.). Further, phased testing may allow for more accurate determination of the date of infection. Such information may be helpful to understanding the progression of the infection and also to implement mitigation efforts such as contact tracing.

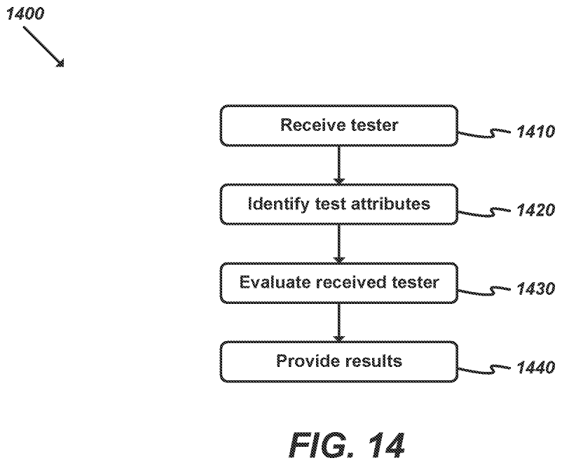

FIG. 14 illustrates a flow chart of an exemplary process 1400 that provides a test result using tester 100 and analyzer 300 (or another external device such as user device 200). The process may generate and/or interpret test results. Further, the process may communicate such test results to various entities. The process may be performed when a sample has been collected at a tester 100. In some embodiments, process 1400 may be performed by analyzer 300 or user device 200. Tester 100 may perform a complementary process.

In some cases, a tester 100 may be supplied to a subject. For instance, a request for a tester 100 or test kit 1200 may be received from a subject or other user at a device such as analyzer 300. The analyzer 300 may house a number of testers 100 or test kits 1200 for a range of antigens that are able to be provided upon request. A tester 100 may be otherwise obtained by a subject in any number of ways, such as via a prescription, over-the-counter purchase, etc.

As shown, process 1400 may include receiving (at 1410) a tester 100. The tester 100 may be received after a sample has been collected and/or a test has otherwise been at least partially implemented (e.g., by cleaning a fingertip before a blood sample is collected by a device such as analyzer 300). A dedicated device, such as analyzer 300, may include a slot or other input that may be able to accept tester 100 or a portion thereof (e.g., test cartridge 420).

In some embodiments, process 1400 may receive or capture data from tester 100, rather than receiving the tester 100 itself. A tester 100 may include a communication module 440 that is able to wirelessly transmit data to analyzer 300. As another example, tester 100 may include various connectors or contacts that may couple complementary connectors or contacts associated with analyzer 300. As still another example, user device 200 or analyzer 300 may capture an image of the tester 100, or a portion thereof (e.g., the face or other area including response areas or other relevant information).

Process 1400 may include identifying (at 1420) test attributes. Test attributes may include attributes related to the tester 100 (e.g., serial number, test type, sample type, output type, etc.), subject (e.g., name, patient number, employee ID, etc.), test (e.g., virus or antigen of interest, test conditions or criteria, etc.), analyzer 300 (e.g., serial number, type, etc.), user device 200 (e.g., application name and version, serial number, type, etc.), and/or other relevant attributes or information (e.g., timestamp, calibration information, etc.). For instance, an image including labeling 550 and/or other informational or communicative elements 560 may be analyzed to extract various test attributes. As another example, test attributes may be received from a practitioner, subject, or other user.

The process may include evaluating (at 1430) the received tester. Such evaluation may include determining whether the test conditions or criteria have been satisfied. Such a determination may be made based on information received from the tester 100 or other source. In some cases, analyzer 300 may implement at least a portion of the test and/or ensure that various conditions have been satisfied. For instance, analyzer 300 may collect a sample and verify that the collected amount is sufficient for the test. As another example, if a wait time is associated with a test (e.g., a delay after a specimen is received to allow for mixing with a buffer solution and/or wicking to a response area), the analyzer 300 may implement such a wait time.

Evaluation may further include determining a result of the test. Based on the test attributes and/or test data received from tester 100, process 1400 may generate a result or output. For instance, tester 100 may include response areas 520-540 that are able to indicate whether a subject has been infected, and, if infected, indicate a stage of progression from among the various stages of an infection.

Different testers 100 and/or associated tests may provide various different outputs and/or data. For instance, some tests may be associated with multiple discrete values, while other tests may be associated with a continuous output (e.g., a value proportional to concentration percentage).

In some embodiments, analyzer 300 may evaluate (and/or otherwise manipulate) the sample itself (or a portion thereof). For instance, if a blood or other specimen is collected at analyzer 300, the analyzer 300 may add buffer solution and apply some portion of the specimen to one or more response areas or other evaluation elements (e.g., sensors) to generate the test data.

As shown, process 1400 may include providing (at 1440) the test results. Such results may be provided via a user interface or other appropriate resource (e.g., by sending the results in an email or text message). Results may be provided in various ways, depending on the type of test, detection capabilities of the tester 100, analyzer 300, and/or user device 200 and/or other relevant attributes. For instance, results for a tester 100 such as tester 500 may be provided as a set of discrete values (e.g., “negative”, “positive, early stage”, “positive, late stage”, “positive, concluded stage”, etc.). As another example, ranges or values may be provided as test results (e.g., “IgM>70% and IgG undetected”, “IgM>70% and IgG>70%”, “IgG>70% and IgM undetected”, etc.).

Different types of test results may be provided to different types of users. For instance, a subject may receive a simple “positive” or “negative” result, while a practitioner may receive a message indicating measured values or ranges, stage of progression, etc.

Results may be provided to various different resources. For instance, results may be sent to a server or other resource for storage and/or future use and analysis. As another example, results may be sent to a subject, practitioner, employer, etc., as appropriate. In some cases, results may be anonymized such that personal information is not disclosed.

Some embodiments may collect other information and associate the information to a subject, test, facility, etc. For instance, body temperature may be measured by a device such as analyzer 300 and the measurements may be stored with test results for analysis (e.g., to better estimate stage of progression). As another example, for a positive test result, analyzer 300 or user device 200 may collect symptom information from a user (e.g., by providing a list of symptoms for selection). As still another example, analyzer 300 or user device 200 may collect contact tracing information from a subject associated with a positive test result.

One of ordinary skill in the art will recognize that processes 1300-1400 may be implemented in various different ways without departing from the scope of the disclosure. For instance, the elements may be implemented in a different order than shown. As another example, some embodiments may include additional elements or omit various listed elements. Elements or sets of elements may be performed iteratively and/or based on satisfaction of some performance criteria. Non-dependent elements may be performed in parallel.

The processes and modules described above may be at least partially implemented as software processes that may be specified as one or more sets of instructions recorded on a non-transitory storage medium. These instructions may be executed by one or more computational element(s) (e.g., microprocessors, microcontrollers, digital signal processors (DSPs), application-specific integrated circuits (ASICs), field programmable gate arrays (FPGAs), other processors, etc.) that may be included in various appropriate devices in order to perform actions specified by the instructions.

As used herein, the terms “computer-readable medium” and “non-transitory storage medium” are entirely restricted to tangible, physical objects that store information in a form that is readable by electronic devices.

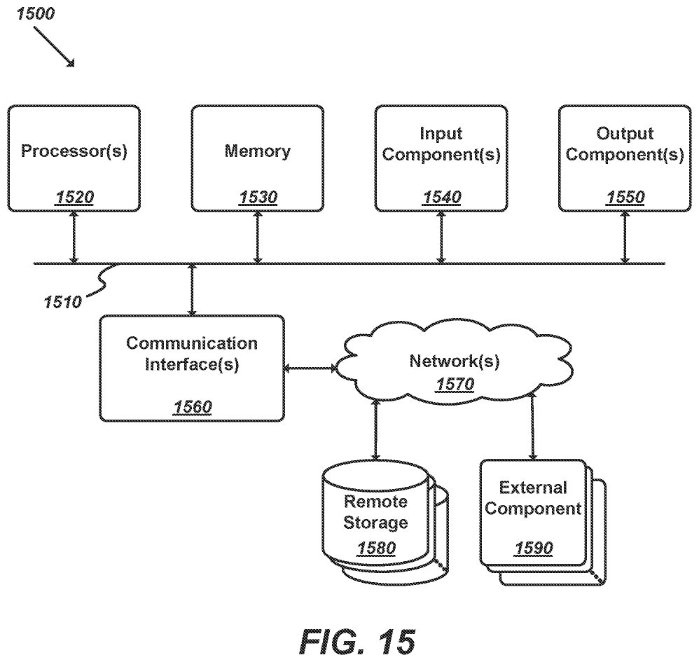

FIG. 15 illustrates a schematic block diagram of an exemplary device (or system or devices) 1500 used to implement some embodiments. For example, the systems described above in reference to FIG. 2 or FIG. 3 may be at least partially implemented using device 1500. As another example, the devices described above in reference to FIG. 1-FIG. 10 may be at least partially implemented using device 1500. As still another example, the processes described in reference to FIG. 13 and FIG. 14 may be at least partially implemented using device 1500.

Device 1500 may be implemented using various appropriate elements and/or sub-devices. For instance, device 1500 may be implemented using one or more personal computers (PCs), servers, mobile devices (e.g., smartphones), tablet devices, wearable devices, and/or any other appropriate devices. The various devices may work alone (e.g., device 1500 may be implemented as a single smartphone) or in conjunction (e.g., some components of the device 1500 may be provided by a mobile device while other components are provided by a server).

As shown, device 1500 may include at least one communication bus 1510, one or more processors 1520, memory 1530, input components 1540, output components 1550, and one or more communication interfaces 1560.

Bus 1510 may include various communication pathways that allow communication among the components of device 1500. Processor 1520 may include a processor, microprocessor, microcontroller, digital signal processor, logic circuitry, and/or other appropriate processing components that may be able to interpret and execute instructions and/or otherwise manipulate data. Memory 1530 may include dynamic and/or non-volatile memory structures and/or devices that may store data and/or instructions for use by other components of device 1500. Such a memory device 1530 may include space within a single physical memory device or spread across multiple physical memory devices.

Input components 1540 may include elements that allow a user to communicate information to the computer system and/or manipulate various operations of the system. The input components may include keyboards, cursor control devices, audio input devices and/or video input devices, touchscreens, motion sensors, etc. Output components 1550 may include displays, touchscreens, audio elements such as speakers, indicators such as light-emitting diodes (LEDs), printers, haptic or other sensory elements, etc. Some or all of the input and/or output components may be wirelessly or optically connected to the device 1500.

Device 1500 may include one or more communication interfaces 1560 that are able to connect to one or more networks 1570 or other communication pathways. For example, device 1500 may be coupled to a web server on the Internet such that a web browser executing on device 1500 may interact with the web server as a user interacts with an interface that operates in the web browser. Device 1500 may be able to access one or more remote storages 1580 and one or more external components 1590 through the communication interface 1560 and network 1570. The communication interface(s) 1560 may include one or more application programming interfaces (APIs) that may allow the device 1500 to access remote systems and/or storages and also may allow remote systems and/or storages to access device 1500 (or elements thereof).

It should be recognized by one of ordinary skill in the art that any or all of the components of computer system 1500 may be used in conjunction with some embodiments. Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

Device 1500 may perform various operations in response to processor 1520 executing software instructions stored in a computer-readable medium, such as memory 1530. Such operations may include manipulations of the output components 1550 (e.g., display of information, haptic feedback, audio outputs, etc.), communication interface 1560 (e.g., establishing a communication channel with another device or component, sending and/or receiving sets of messages, etc.), and/or other components of device 1500.

The software instructions may be read into memory 1530 from another computer-readable medium or from another device. The software instructions stored in memory 1530 may cause processor 1520 to perform processes described herein. Alternatively, hardwired circuitry and/or dedicated components (e.g., logic circuitry, ASICs, FPGAs, etc.) may be used in place of or in combination with software instructions to implement processes described herein. Thus, implementations described herein are not limited to any specific combination of hardware circuitry and software.

The actual software code or specialized control hardware used to implement an embodiment is not limiting of the embodiment. Thus, the operation and behavior of the embodiment has been described without reference to the specific software code, it being understood that software and control hardware may be implemented based on the description herein.

While certain connections or devices are shown, in practice additional, fewer, or different connections or devices may be used. Furthermore, while various devices and networks are shown separately, in practice the functionality of multiple devices may be provided by a single device or the functionality of one device may be provided by multiple devices. In addition, multiple instantiations of the illustrated networks may be included in a single network, or a particular network may include multiple networks. While some devices are shown as communicating with a network, some such devices may be incorporated, in whole or in part, as a part of the network.

Some implementations are described herein in conjunction with thresholds. To the extent that the term “greater than” (or similar terms) is used herein to describe a relationship of a value to a threshold, it is to be understood that the term “greater than or equal to” (or similar terms) could be similarly contemplated, even if not explicitly stated. Similarly, to the extent that the term “less than” (or similar terms) is used herein to describe a relationship of a value to a threshold, it is to be understood that the term “less than or equal to” (or similar terms) could be similarly contemplated, even if not explicitly stated. Further, the term “satisfying,” when used in relation to a threshold, may refer to “being greater than a threshold,” “being greater than or equal to a threshold,” “being less than a threshold,” “being less than or equal to a threshold,” or other similar terms, depending on the appropriate context.

No element, act, or instruction used in the present application should be construed as critical or essential unless explicitly described as such. An instance of the use of the term “and,” as used herein, does not necessarily preclude the interpretation that the phrase “and/or” was intended in that instance. Similarly, an instance of the use of the term “or,” as used herein, does not necessarily preclude the interpretation that the phrase “and/or” was intended in that instance. Also, as used herein, the article “a” is intended to include one or more items and may be used interchangeably with the phrase “one or more.” Where only one item is intended, the terms “one,” “single,” “only,” or similar language is used. Further, the phrase “based on” is intended to mean “based, at least in part, on” unless explicitly stated otherwise.

The foregoing relates to illustrative details of exemplary embodiments and modifications may be made without departing from the scope of the disclosure. Even though particular combinations of features are recited in the claims and/or disclosed in the specification, these combinations are not intended to limit the possible implementations of the disclosure. In fact, many of these features may be combined in ways not specifically recited in the claims and/or disclosed in the specification. For instance, although each dependent claim listed below may directly depend on only one other claim, the disclosure of the possible implementations includes each dependent claim in combination with every other claim in the claim set.

We claim:

1. A tester, comprising:
a sample collection area that receives a fluid sample;
a first result indication area that provides a first visual indicator of a valid test result when a set of test conditions is satisfied;
a second result indication area that provides a second visual indicator when a first test criteria is satisfied; and
a third result indication area that provides a third visual indicator when a second test criteria is satisfied.

2. The tester of claim 1, wherein the set of test conditions comprises a minimum sample amount, a specimen type, a minimum elapsed processing time, or a minimum threshold value for a measured value associated with the fluid sample.

3. The tester of claim 1, wherein each of the first result indication area, second result indication area, and third result indication area receive a portion of the fluid sample via one or more absorbent portions that wick the fluid sample from the sample collection area.

4. The tester of claim 1, wherein the fluid sample is blood.

5. The tester of claim 1 further comprising an aqueous buffer solution comprising a weak acid and a weak conjugate base, wherein the aqueous buffer solution is combined with at least a portion of the fluid sample.

6. The tester of claim 1, wherein the first test criteria comprise a first concentration threshold associated with Immunoglobin G and the second test criteria comprise a second concentration threshold associated with Immunoglobin M.

7. The tester of claim 1, wherein the tester is associated with a Severe Acute Respiratory Syndrome Coronavirus-2 (SARS-CoV-2) infection test.

8. A test kit comprising:
a first tester, comprising:
a sample collection area that receives a fluid sample;
a first result indication area that provides a first visual indicator of a valid test result when a set of test conditions is satisfied;
a second result indication area that provides a second visual indicator when a first test criteria is satisfied; and
a third result indication area that provides a third visual indicator when a second test criteria is satisfied;
usage instructions;
a buffer solution; and
a lancet.

9. The test kit of claim 8 further comprising a pipette.

10. The test kit of claim 8 further comprising:
an alcohol swab;
a set of disposable gloves; and
a refuse bag.

11. The test kit of claim 8, wherein the set of test conditions comprises a minimum sample amount, a specimen type, a minimum elapsed processing time, or a minimum threshold value for a measured value associated with the fluid sample.

12. The test kit of claim 8, wherein each of the first result indication area, second result indication area, and third result indication area receive a portion of the fluid sample via one or more absorbent portions that wick the fluid sample from the sample collection area.

13. The test kit of claim 8, wherein the fluid sample is blood, and the buffer solution is an aqueous buffer solution comprising a weak acid and a weak conjugate base, wherein the buffer solution is combined with at least a portion of the fluid sample.

14. The test kit of claim 8, wherein:
the first test criteria comprise a first concentration threshold associated with Immunoglobin G and the second test criteria comprise a second concentration threshold associated with Immunoglobin M, and
the tester is associated with a Severe Acute Respiratory Syndrome Coronavirus-2 (SARS-CoV-2) infection test.

15. A method comprising:
receiving a fluid sample at a tester device;
processing the received fluid sample;
evaluating the processed fluid sample; and
providing a test result based on the evaluation of the processed sample.

16. The method of claim 15, wherein the fluid sample comprises blood and processing the received sample comprises combining an aqueous buffer solution comprising a weak acid and a weak conjugate base with at least a portion of the fluid sample.

17. The method of claim 15, wherein providing the test result comprises providing a visual indicator if a set of test conditions is satisfied.

18. The method of claim 15, wherein evaluating the processed sample comprises:
determining whether a concentration of Immunoglobin G in the processed sample exceeds a first concentration threshold; and
determining whether a concentration of Immunoglobin M in the processed sample exceeds a second concentration threshold.

19. The method of claim 18, wherein providing the test result comprises:
providing a first visual indicator if the concentration of Immunoglobin G in the processed sample exceeds a first concentration threshold; and
providing a second visual indicator if the concentration of Immunoglobin M in the processed sample exceeds a second concentration threshold.

20. The method of claim 15, wherein the tester device is associated with a Severe Acute Respiratory Syndrome Coronavirus-2 (SARS-CoV-2) infection test.
